(19) United States
(12) Patent Application Publication
(10) Pub. No.: US 2024/0252511 A1
(43) Pub. Date: Aug. 1, 2024

(54) METHODS OF TREATING DEPRESSION AND ANXIETY

(71) Applicant: Woolsey Pharmaceuticals, Inc., St. Petersburg, FL (US)

(72) Inventors: Thomas MACALLISTER, Arlington, VA (US); Sven JACOBSON, New York, NY (US)

(21) Appl. No.: 18/560,763

(22) PCT Filed: May 16, 2022
(86) PCT No.: PCT/US2022/029402

§ 371 Date: Nov. 14, 2023

Related U.S. Application Data
(60) Provisional application No. 63/190,512, filed on May 19, 2021.

(51) Int. Cl. A61K 31/551

(52) U.S. Cl. CPC A61K 31/551

(57) ABSTRACT

The invention relates to the treatment of patients with depression and or anxiety with low doses of rho kinase inhibitors. The maximum dose of a rho kinase inhibitor, based on fasudil hydrochloride as an exemplary agent, is less than 60 mg per day based on an immediate release formulation. Comparable dosing with other inhibitors is selected based on molar equivalents and/or rho kinase binding affinities. Treatable patients have one or more depressive disorders and/or one or more anxiety disorders.

### CROSS-REFERENCE TO RELATED APPLICATIONS

This patent application is the National Stage entry of International Application No. PCT/US2022/029402, filed 16 May 2022, which claims the benefit of U.S. Provisional Patent Application No. 63/190,512, filed May 19, 2021. The disclosure of the priority application is incorporated in its entirety herein by reference.

### BACKGROUND OF THE INVENTION

While depression and anxiety are considered different conditions, they coexist in many patients and have similar treatments. Anxiety may occur as a symptom of major depression. In addition, depression is commonly triggered by an anxiety disorder, such as generalized anxiety disorder, panic disorder or separation anxiety disorder. About half of the people with anxiety disorder or clinical depression have both. Antidepressant drugs are generally efficacious in treating anxiety and are often considered first-line therapy.

Depression disorders refer to any of several disorders associated with periods of depression. The Diagnostic and Statistical Manual of Mental Disorders, Fifth Edition (DSM-5) categorizes some depressive disorders by specific symptoms: major depressive disorder (aka major depression); persistent depressive disorder (aka dysthymia); and other specified or unspecified depressive disorder. It further categorizes some by etiology: premenstrual dysphoric disorder; depressive disorder due to another medical condition; substance/medication-induced depressive disorder.

The cause of depression is unknown, but heredity appears to be an important component. Depression is more common among 1st-degree relatives of depressed patients, and concordance between identical twins is high. Imbalances in neurotransmitter levels have been implicated, including abnormal regulation of cholinergic, catecholaminergic (noradrenergic or dopaminergic), glutamatergic, and serotonergic (5-hydroxytryptamine) neurotransmission. Many antidepressants act on these pathways. Psychosocial factors seem important. Major life stresses often precede episodes of major depression. Depression may also develop in people with other mental disorders. Women are at higher risk, possibly due to higher levels of monoamine oxidase, higher rates of thyroid dysfunction, endocrine differences and specifically changes that occur with menstruation and at menopause.

Depressive disorders may accompany various physical disorders, including thyroid disorders, adrenal gland disorders, benign and malignant brain tumors, stroke, AIDS, Parkinson disease, and multiple sclerosis. Certain drugs, such as corticosteroids, some beta-blockers, interferon, and reserpine, can also result in depressive disorders. Abuse of some recreational drugs (e.g., alcohol, amphetamines) also can lead to depression.

Anxiety disorders are characterized by frequent and intense, excessive and persistent worry and fear about everyday situations. They may involve repeated episodes of sudden feelings of intense anxiety and fear or terror that reach a peak within minutes (panic attacks). These feelings of anxiety and panic are such that they interfere with daily activities, are difficult to control, are out of proportion to the actual danger, and can be long lasting. Examples of anxiety disorders include generalized anxiety disorder, social anxiety disorder (social phobia), specific phobias and separation anxiety disorder. You can have more than one anxiety disorder. Sometimes anxiety results from a medical condition that needs treatment.

Classes of antidepressant drugs, which may be used to treat depression and anxiety, include selective serotonin reuptake inhibitors, serotonin-norepinephrine reuptake inhibitors, serotonin modulators and stimulators, serotonin antagonists and reuptake inhibitors, norepinephrine reuptake inhibitors, norepinephrine-dopamine reuptake inhibitors, tricyclic antidepressants, tetracyclic antidepressants, monoamine oxidase inhibitors, and NMDA receptor antagonists. In addition to the antidepressants, other common anxiolytics include barbiturates, benzodiazepines, carbamates, antihistamines, opioids and sympatholytics (adrenergic modulators), among others. Treatments are often used in combinations and it is a constant balance to manage efficacy with side effects, which may multiply with the addition of similar classes of drugs together. A need exists for new classes of molecules that can be used to treat depression and anxiety that can provide relief, but do not exacerbate the existing challenge of managing side effects.

While there may be interventions that seem promising in animal models of depression-like and anxiety-like behavior, a grave degree of skepticism should be applied in interpreting animal data. Even aside from the obvious issues of differences in brain complexity between animals (e.g., rodents) and humans, many of the existing models bear only a passing resemblance to the human condition. Many things can cause depression and anxiety in animals and many putative drugs can reduce depression-like and anxiety-like behaviors stemming from such conditions, but the underlying pathophysiology and chain of causation of depression and anxiety are unknown and it is there that a disease-modifying intervention must act. It is crucial, therefore, that animal models, with their known deficiencies in the best of cases, as closely resemble the human disease as possible, in both pathology and clinical presentation.

There are a number of publications looking at the use of rho kinase inhibitors in various animal models of depressive-like and anxiety-like behavior. The established models are deficient even in their basic properties. Some models involve the direct induction of acute restraint stress (Saitoh 2006; Garcia-Rojo 2017) or immobility (which is considered to reflect a measure of behavioral despair), or employ forced swim tests (FSTs) (Shapiro 2019; Yankeletitch-Yahav 2015; Garcia-Rojo 2017), while others withhold food from rodents and measure latency to approach food, supposedly a test predictive of therapeutic onset of an intervention (Shapiro 2019). Still other models measure physiological response (in the amygdala) when exposing animals to new objects (Sarowar 2017) or utilize classical fear conditioning (e.g., footshock) (Lamprecht 2002), social defeat stress (e.g., cage placement with aggressive animals) (Fox 2020; Francis 2019; Li 2017) or animal-on-animal attacks, witnessed by other animals (Nakatake 2020), and at least one model directly induces traumatic brain injury (TBI) using an electromagnetic impact device (Piao 2019). Another study even suggests that exposure to fasudil, a rho kinase inhibitor, induces anxiety-like behaviors in adult mice (Greathouse 2019). While these models may exhibit certain depression- or anxiety-like properties, they are merely behavioral models and cannot predict treatment of depression and anxiety themselves.

Specifically, the animal models do not faithfully recapitulate human disease, partly due to species differences in neuroanatomy (Sasaguri 2017) and partly due to the deficient basic pathological bases of the models, described above. Yankelevitch-Yahav 2015 expressed reservations about FSTs, questioning whether immobility in the FST and depression share the same long-term adaptive changes in neuronal circuitry that underlie the effects of antidepressants in humans and noting that, in contrast with FST test subjects, real-life patients need to be treated for at least several weeks before they experience any relief from their symptoms. Piao 2019 administered subcutaneous infusions of fasudil (25 mg/kg/day for 7 days) to mice experiencing TBI but monitored only changes in hippocampal GLT1 and GLAST. Saitoh 2006 used intracerebroventricular infusion of trans-4-[(1R)-1-aminoethyl]-N-4-pyridinylcyclohexanecarboxamide (Y-27632), a ROCK inhibitor, and observed effects on anxiety-related behaviors due to elevated plus-maze tests and Y-maze tests but expressed no findings on actual anxiety or depression, Roman-Albasini 2020 administered intraperitoneal injections of fasudil (10 mg/kg/day) to rats, but only suggests that fasudil may prevent chronic stress-altered behaviors.

Some publications use unrealistic routes of administration (e.g., intracerebroventricular injection or intraperitoneal injection) and many do not use appropriate dosing. In this regard, standard formulas exist for converting doses used in animals to the same dose in humans. Human equivalent dose can be calculated, for example, using Table 1 of Nair & Jacob, J Basic Clin Pharm. 7:27-31 (2016), which are the same conversions used by the US FDA; such methods utilize body surface area (BSA)-based dose determinations. Yet such methods are not without their shortcomings. Vieira 2019, for example, notes that BSA-based dose determination is poorly correlated with the pharmacokinetics of many classes of drugs, often leading to suboptimal dosing, including both inefficacious underdosing, and potentially lethal overdosing and that BSA calculations fail to account for abnormal body sizes or genetic variation across populations.

Based on currently-available but deficient animal modeling, different therapeutic strategies targeting depression-like and anxiety-like behaviors have been tested, but have failed to show any beneficial effects in humans. There exists a significant unmet need to provide new, disease modifying therapies that show benefit in humans, not just animals.

### SUMMARY OF THE INVENTION

The invention contemplates the treatment of depression and/or anxiety with a rho kinase inhibitor. In an embodiment, the rho kinase inhibitor is fasudil and it is administered in a therapeutically effective amount one or more times daily. In a preferred embodiment, fasudil is administered orally in a dose of less than 60 mg per day. In preferred methods, subjects have a depressive order that may cause prolonged cognitive, psychomotor, and/or other types of dysfunction (e.g., poor concentration, fatigue, loss of sexual desire, loss of interest or pleasure in nearly all activities that were previously enjoyed, sleep disturbances), as well as a depressed mood. In preferred methods, subjects experience one or more depressive orders including major depression, persistent depressive disorder, bipolar disorder, premenstrual dysphoric disorder, and other depressive disorder. In preferred methods, subject experience one or more anxiety disorders including generalized anxiety disorder, panic disorder, phobia-related disorders, separation anxiety disorder, selective mutism, anxiety disorder due to a medical condition, substance-induced anxiety disorder, and other specified anxiety disorder and unspecified anxiety disorder.

The inventive methods further contemplate treating subjects experiencing one or more of the following: depressed mood or loss of interest in activities for most days over at least a two-week or at least a two-year period, depressed mood most of the day, markedly diminished interest or pleasure in all or almost all activities for most of the day, significant (>5%) weight gain or loss or decreased or increased appetite, insomnia or hypersomnia, psychomotor agitation or retardation observed by others, fatigue or loss of energy, feelings of worthlessness or excessive or inappropriate guilt, diminished ability to think or concentrate or indecisiveness, recurrent thoughts of death or suicide, low self-esteem, feelings of hopelessness, episodes of mood ranging from high energy periods to low energy periods, mood and anxiety symptoms related to the menstrual cycle, marked mood swings, marked irritability or anger or increased interpersonal conflicts, marked depressed mood, feeling keyed up or tense, feeling unusually restless, difficulty concentrating because of worry, fear that something awful may happen, feeling of potential loss of control, elevated mood, grandiosity, inflated self-esteem, increased energy or goal-directed activity, excessive talkativeness or pressured speech, racing thoughts or flight of ideas, engaging in pleasurable but risky activities with serious potential consequences, overreaction to perceived criticism or rejection, feelings of leaden paralysis, delusions, hallucinations, severe psychomotor retardation, echolalia, echopraxia, nervousness, irritability, feeling wound-up, feeling on-edge, having muscle tension, difficulty controlling feelings of worry, having a sense of impeding danger, increased heart rate, hyperventilating, sweating, trembling, gastrointestinal problems, unexpected panic attacks, phobia-related disorders, fear of or anxiety toward social or performance situations, fear of using public transportation, fear of being in enclosed spaces, fear of standing in line or being in a crowd, fear of being outside of the home alone, extreme shyness, compulsive traits, withdrawal, clinging behavior, temper tantrums, and misuse of drugs.

In an embodiment, treatment with fasudil results in an improvement in one or more of the conditions described in the preceding paragraph.

In one embodiment, treatment with fasudil results in decreased depression or anxiety compared to that measured in the same subject prior to treatment with fasudil or in a longitudinal cohort of subjects. Treatment effectiveness may be gauged by methods known in the art, including the Hamilton Rating Scale for Depression, Montgomery Åsberg Depression Rating Scale, the Clinical Global Impression—Severity, Hamilton Rating Scale for Anxiety, Columbia-Suicide Severity Rating Scale, the Short Form-36 (SF-36), the Patient Health Questionnaire (PHQ-9), the Sheehan Disability Scale (SDS), and The World Health Organisation-Five Well-Being Index.

In another embodiment, treatment with fasudil results in improved sustained and/or selective mood improvement compared to that measured in the same subject prior to treatment with fasudil or in a longitudinal cohort of subjects.

In one embodiment, the invention contemplates combination therapy with at least one rho kinase inhibitor and at least one known antidepressant or anxiolytic, either by dosing each drug separately or dosing them together in a fixed-dose combination.

In a specific embodiment, the invention contemplates co-administration of fasudil in combination with B-vitamins including B12, B6, B9, antioxidants (e.g., vitamins, C, E, beta-carotene and flavonoids), and omega 3-fatty acids.

In another specific embodiment, the invention contemplates co-administration of fasudil in combination with plant polyphenols such as resveratrol.

### DETAILED DESCRIPTION OF THE INVENTION

The invention is based on the observation that rho kinase (ROCK) inhibitors can be used successfully to manage symptoms of depression and anxiety, either alone or in conjunction with existing pharmaceutical or non-pharmaceutical management approaches.

### ROCK Inhibitors

The inventive methods contemplate the administration of a rho kinase (ROCK) inhibitor in the treatment of a disease or condition. Two mammalian ROCK homologs are known, ROCK1 (aka ROKβ, Rho-kinase β, or p160ROCK) and ROCK2 (aka ROKα) (Nakagawa 1996). In humans, the genes for both ROCK1 and ROCK2 are located on chromosome 18. The two ROCK isoforms share 64% identity in their primary amino acid sequence, whereas the homology in the kinase domain is even higher (92%) (Jacobs 2006; Yamaguchi 2006). Both ROCK isoforms are serine/threonine kinases and have a similar structure.

A large number of pharmacological ROCK inhibitors are known (Feng, LoGrasso, Defert, & Li, 2015). Isoquinoline derivatives are a preferred class of ROCK inhibitors. The isoquinoline derivative fasudil was the first small molecule ROCK inhibitor developed by Asahi Chemical Industry (Tokyo, Japan). The characteristic chemical structure of fasudil consists of an isoquinoline ring, connected via a sulphonyl group to a homopiperazine ring. Fasudil is a potent inhibitor of both ROCK isoforms. In vivo, fasudil is subjected to hepatic metabolism to its active metabolite hydroxyfasudil (aka, M3). Other examples of isoquinoline derived ROCK inhibitors include dimethylfasudil and ripasudil.

Other preferred ROCK inhibitors are based on based on 4-aminopyridine structures. These were first developed by Yoshitomi Pharmaceutical (Uehata et al., 1997) and are exemplified by Y-27632. Still other preferred ROCK inhibitors include indazole, pyrimidine, pyrrolopyridine, pyrazole, benzimidazole, benzothiazole, benzathiophene, benzamide, aminofurazane, quinazoline, and boron derivatives (Feng et al., 2015). Exemplary ROCK inhibitors are below:

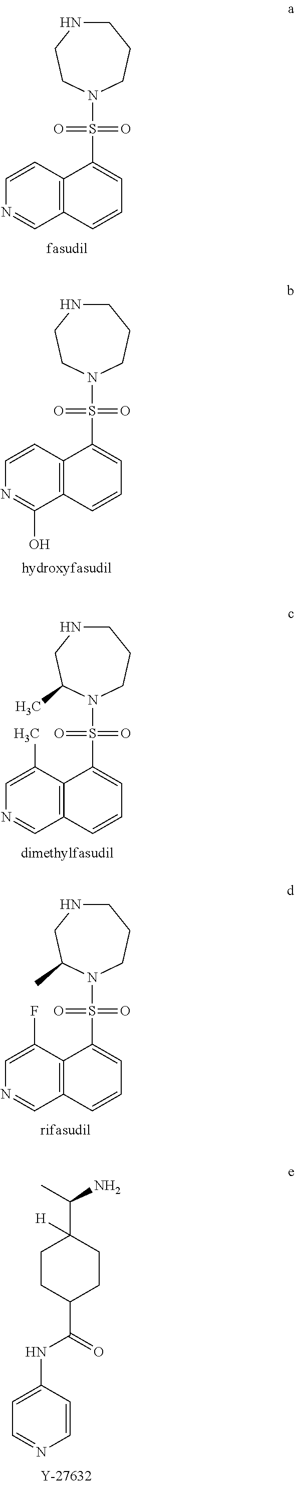 [SMILES: O=S(=O)(c1cccc2cnccc12)N1CCCNCC1 ; O=S(=O)(c1cccc2c(O)nccc12)N1CCCNCC1 ; Cc1cncc2cccc(S(=O)(=O)N3CCCNC[C@@H]3C)c12 ; C[C@H]1CNCCCN1S(=O)(=O)c1cccc2cncc(F)c12 ; C[C@@H](N)C1CCC(C(=O)Nc2ccncc2)CC1]

ROCK inhibitors according to the invention may have more selective activity for either ROCK1 or ROCK2 and will usually have varying levels of activity on PKA, PKG, PKC, and MLCK. Some ROCK inhibitors may be highly specific for ROCK1 or ROCK2 and have much lower activity against PKA, PKG, PKC, and MLCK.

A particularly preferred ROCK inhibitor is fasudil. Fasudil may exist as a free base or salt and may be in the form of a hydrate, such as a hemihydrate.

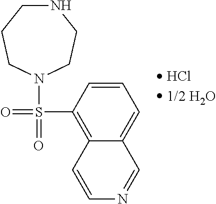 [SMILES: O=S(=O)(c1cccc2cnccc12)N1CCCNCC1]

Hexahydro-1-(5-isoquinolinesulfonyl)-1H-1,4-diazepine monohydrochloride hemihydrate

Fasudil is a selective inhibitor of protein kinases, such as ROCK, PKC and MLCK and treatment results in a potent relaxation of vascular smooth muscle, resulting in enhanced blood flow (Shibuya 2001). In an embodiment, fasudil is a potent inhibitor of the ROCK1 isoform. In another embodiment, fasudil is a potent inhibitor of the ROCK2 isoform. A particularly important mediator of vasospasm, ROCK induces vasoconstriction by phosphorylating the myosin-binding subunit of myosin light chain (MLC) phosphatase, thus decreasing MLC phosphatase activity and enhancing vascular smooth muscle contraction. Moreover, there is evidence that fasudil increases endothelial nitric oxide synthase (eNOS) expression by stabilizing eNOS mRNA, which contributes to an increase in the level of the potent vasodilator nitric oxide (NO), thereby enhancing vasodilation (Chen 2013).

Fasudil has a short half-life of about 25 minutes, but it is substantially converted in vivo to its 1 hydroxy (M3) metabolite. M3 has similar effects to its fasudil parent molecule, with slightly enhanced activity and a half-life of about 8 hours (Shibuya 2001). Thus, M3 is likely responsible for the bulk of the in vivo pharmacological activity of the molecule and its use is contemplated to be interchangeable with fasudil on a molar basis. M3 exists as two tautomers, depicted below:

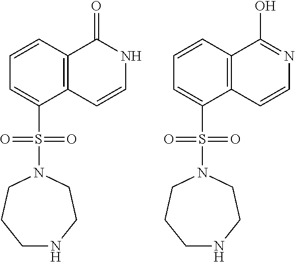 [SMILES: O=c1[nH]ccc2c(S(=O)(=O)N3CCCNCC3)cccc12 ; O=S(=O)(c1cccc2c(O)nccc12)N1CCCNCC1]

The ROCK inhibitors used in the invention, such as fasudil, include pharmaceutically acceptable salts and hydrates. Salts that may be formed via reaction with inorganic and organic acid. Those inorganic and organic acids are included as following: hydrochloric acid, hydrobromide acid, hydriodic acid, sulphuric acid, nitric acid, phosphoric acid, acetic acid, maleic acid, maleic acid, maleic acid, oxalic acid, oxalic acid, tartaric acid, malic acid, mandelic acid, trifluoroacetic acid, pantothenic acid, methane sulfonic acid, or para-toluenesulfonic acid.

### Pharmaceutical Compositions

Pharmaceutical compositions of ROCK inhibitors usable in the invention are generally oral and may be in the form of tablets or capsules and may be immediate-release formulations or may be controlled- or extended-release formulations, which may contain pharmaceutically acceptable excipients, such as corn starch, mannitol, povidone, magnesium stearate, talc, cellulose, methylcellulose, carboxymethylcellulose and similar substances. A pharmaceutical composition comprising a ROCK inhibitor and/or a salt thereof may comprise one or more pharmaceutically acceptable excipients, which are known in the art. Formulations include oral films, orally disintegrating tablets, effervescent tablets and granules or beads that can be sprinkled on food or mixed with liquid as a slurry or poured directly into the mouth to be washed down.

Pharmaceutical compositions containing ROCK inhibitors, salts and hydrates thereof can be prepared by any method known in the art of pharmaceutics. In general, such preparatory methods include the steps of bringing a ROCK inhibitor or a pharmaceutically acceptable salt thereof into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. The composition used in accordance with the methods of the present invention may comprise between 0.001% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

In certain embodiments, the pharmaceutical composition used in the methods of the present invention may comprise a diluent. Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

In certain embodiments, the pharmaceutical composition used in the methods of the present invention may comprise a granulating and/or dispersing agent. Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM), sodium lauryl sulfate, quaternary ammonium compounds, and mixtures thereof.

In certain embodiments, the pharmaceutical composition used in the methods of the present invention may comprise a binding agent. Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM.RTM.), and larch arabogalactan), alginates, polyethylene oxide, polyethylene glycol, inorganic calcium salts, silicic acid, polymethacrylates, waxes, water, alcohol, and/or mixtures thereof.

In certain embodiments, the pharmaceutical composition used in the methods of the present invention may comprise a preservative. Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

In certain embodiments, the pharmaceutical composition used in the methods of the present invention may comprise an antioxidant. Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

In certain embodiments, the pharmaceutical composition used in the methods of the present invention may comprise a chelating agent. Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

In certain embodiments, the pharmaceutical composition may comprise a buffering agent together with the ROCK inhibitor or the salt thereof. Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

In certain embodiments, the pharmaceutical composition used in the methods of the present invention may comprise a lubricating agent. Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

In other embodiments, the pharmaceutical composition of containing a ROCK inhibitor or salt thereof will be administered as a liquid dosage form. Liquid dosage forms for oral and parenteral administration include pharmaceutically acceptable emulsions, microemulsions, solutions, suspensions, syrups, and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor™, alcohols, oils, modified oils, glycols, polysorbates, cyclodextrins, polymers, and mixtures thereof.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Some compositions of the invention relate to extended- or controlled-release formulations. These may be, for example, diffusion-controlled products, dissolution-controlled products, erosion products, osmotic pump systems or ionic resin systems. Diffusion-controlled products comprise a water-insoluble polymer which controls the flow of water and the subsequent egress of dissolved drug from the dosage from. Dissolution-controlled products control the rate of dissolution of the drug by using a polymer that slowly solubilizes or by microencapsulation of the drug—using varying thicknesses to control release. Erosion products control release of drug by the erosion rate of a carrier matrix. Osmotic pump systems release a drug based on the constant inflow of water across a semi permeable membrane into a reservoir which contains an osmotic agent. Ion exchange resins can be used to bind drugs such that, when ingested, the release of drug is determined by the ionic environment within the gastrointestinal tract.

### Methods of Treatment

### Conditions

The inventive methods contemplate using a rho kinase inhibitor to treat one or more conditions associated with depression and/or anxiety. Conditions selected for treatment are differentiated from normal mood swings due to the fact that symptoms interfere with the normal functioning of the patient, physically, occupationally and/or socially. Thus, merely feeling low, discouraged or disappointed about the normal set-backs of life (e.g., financial distress, natural disaster, serious illness) or losses (e.g., death of a loved one) are generally not treatable according to the invention. Unlike the disorders treatable in accordance with the invention, these normal negative feelings tend to occur in waves tied to reminders of the inciting event, resolve when circumstances improve, may be interspersed with periods of positive emotion, are not accompanied by pervasive feelings of worthlessness and self-loathing. They tend to last short periods of time, like days, rather than weeks or months, and do not tend to elicit suicidal thoughts and or induce prolonged loss of function.

Depressive disorders treatable according to the invention, on the other hand, cause prolonged cognitive, psychomotor, and/or other types of dysfunction (e.g., poor concentration, fatigue, loss of sexual desire, loss of interest or pleasure in nearly all activities that were previously enjoyed, sleep disturbances), as well as a depressed mood. Suicidal ideation is common, and patients may attempt suicide. Anxiety frequently coexists. Patients are more likely to abuse alcohol or other recreational drugs. They are also more likely to become heavy smokers and to neglect their health, increasing the risk of other disorders. Compounding this, depression may reduce protective immune responses and increase the risk of cardiovascular disorders, like myocardial infarctions and stroke.

Depressive disorders treatable according to the invention include major depression, persistent depressive disorder, bipolar disorder, premenstrual dysphoric disorder, other depressive disorder. Major depression and persistent depressive disorder treatable according to the invention may be further categorized as anxious distress, mixed feature, melancholic, atypical, psychotic, catatonic, peripartum (or postpartum), and seasonal pattern.

Subjects with major depression (unipolar disorder, clinical depression or major depressive disorder) will feel depressed most of the time for most days of the week. Major depression is usually considered present when the subject (a) has a depressed mood or loss of interest in activities for most days over at least a two-week period and (b) has five or more of the following symptoms: depressed mood most of the day; markedly diminished interest or pleasure in all or almost all activities for most of the day; significant (>5%) weight gain or loss or decreased or increased appetite (nutrition may be significantly impaired); insomnia (often sleep-maintenance insomnia) or hypersomnia; psychomotor agitation or retardation observed by others; fatigue or loss of energy; feelings of worthlessness or excessive or inappropriate guilt; diminished ability to think or concentrate or indecisiveness; and recurrent thoughts of death or suicide, a suicide attempt, or a specific plan for committing suicide.

To the observer, patients with major depression may appear miserable or tearful, have furrowed brows, frown often, display slumped posture, avoid eye contact, lack facial expression, show little body movement, and display speech changes like a soft voice, lack of prosody, the use of monosyllabic words. Some depressed patients neglect personal hygiene, loved ones or pets.

Persistent depressive disorder (PDD) includes chronic major depressive disorder and dysthymic disorder (low-grade persistent depression). It requires that depressive symptoms that persist for at least 2 years without remission, but the severity may vary. PDD may or may not fall within the definition of major depression and the number of symptoms may fluctuate. PDD patients will have had a depressed mood for most of the day for more days than not for at least 2 years and at least two of the following symptoms: poor appetite or overeating; insomnia or hypersomnia; low energy or fatigue; low self-esteem; poor concentration or difficulty making decisions; and feelings of hopelessness.

Affected patients may appear gloomy, pessimistic, humorless, passive, lethargic, introverted, hypercritical of self and others, and complaining. Patients with PDD are also more likely to have underlying anxiety disorders, substance use disorders, or personality (ie, borderline personality) disorders.

Patients with bipolar disorder (aka “manic depression”) display episodes of mood ranging from high energy, “up” periods to low energy “depressive” periods. When in the depressive phase, the symptoms of major depression are present.

Premenstrual dysphoric disorder involves mood and anxiety symptoms that are clearly related to the menstrual cycle, with onset during the premenstrual phase and a symptom-free interval after menstruation. Symptoms must be present during most menstrual cycles during the past year.

Manifestations are similar to those of premenstrual syndrome but are more severe, causing clinically significant distress and/or marked impairment of social or occupational functioning. The disorder may begin any time after menarche; it may worsen as menopause approaches but ceases after menopause. Prevalence is estimated at 2 to 6% of menstruating women in a given 12-month interval.

For diagnosis of premenstrual dysphoric disorder, patients must have ≥5 symptoms during the week before menstruation. Symptoms must begin to remit within a few days after onset of menses and become minimal or absent in the week after menstruation. Symptoms must include ≥1 of the following: marked mood swings (e.g., suddenly feeling sad or tearful); marked irritability or anger or increased interpersonal conflicts; marked depressed mood, feelings of hopelessness, or self-deprecating thoughts; and marked anxiety, tension, or an on-edge feeling. In addition, at least one of the following must be present: decreased interest in usual activities; difficulty concentrating; low energy or fatigue; marked change in appetite, overeating, or specific food cravings; hypersomnia or insomnia; and feeling overwhelmed or out of control. Physical symptoms such as breast tenderness or swelling, joint or muscle pain, a feeling of being bloated, and weight gain.

Other depressive (specified or unspecified) disorder includes clusters of symptoms with characteristics of a depressive disorder that do not meet the full criteria for other depressive disorders but that cause clinically significant distress or impairment of functioning. Other depressive disorder includes recurrent periods of dysphoria with at least 4 other depressive symptoms that last less than 2 weeks in people who have never met criteria for another mood disorder (e.g., recurrent brief depression) and depressive periods that last longer but that include insufficient symptoms for diagnosis of another depressive disorder.

Major depression and persistent depressive disorder may include one or more “specifiers” that describe additional manifestations during a depressive episode. These include anxious distress, mixed feature, melancholic, atypical, psychotic, catatonic, peripartum (or postpartum), and seasonal pattern.

Anxious distress depression patients are tense and restless, they cannot concentrate because fear that something bad may happen, or they feel that they may lose control of themselves. Anxious distress is formally defined as the presence of at least 2 of the following symptoms: feeling keyed up or tense; feeling unusually restless; difficulty concentrating because of worry; fear that something awful may happen; and feeling of potential loss of control.

The severity of anxious distress is further specified as: mild: (2 symptoms); moderate (3 symptoms); moderate-severe (4-5 symptoms); and severe (4-5 symptoms with motor agitation).

Mixed feature depression (aka “mixed episode,” “mixed state,” or “agitated depression”) patients have symptoms of both depression and mania or hypomania at the same time. A diagnosis formally requires at least 3 manic or hypomanic symptoms most days for at least two weeks. These include elevated mood, grandiosity, inflated self-esteem, increased energy or goal-directed activity, excessive talkativeness or pressured speech, racing thoughts or flight of ideas, decreased need for sleep, and engaging in pleasurable, but risky activities with serious potential consequences (excessive alcohol use, risky sexual behaviors, impulsive spending, etc.). Patients with mixed feature depression are at risk of developing bipolar disorder.

Melancholic depression patients no longer experience much, if any, pleasure or do not respond to pleasurable stimuli. They may be despondent and despairing, feel excessive or inappropriate guilt, or have early morning awakenings, marked psychomotor retardation or agitation, and significant anorexia or weight loss. The following are some typical symptoms: distinctly depressed mood characterized by profound despondency, despair, or emptiness; depression is worse in the morning; early morning waking of at least two hours earlier than normal; psychomotor disturbances of either retardation or agitation; anorexia or weight loss; and excessive or inappropriate guilt.

Atypical depression patients may show temporarily improved mood in response to positive events (e.g., a visit from children). They also have ≥2 of the following: overreaction to perceived criticism or rejection, feelings of leaden paralysis (typically heaviness in the arms or legs), weight gain or increased appetite, and hypersomnia.

Psychotic depression patients have delusions (false beliefs) and/or hallucinations. Delusions often involve having committed unpardonable sins or crimes, harboring incurable or shameful disorders, or being persecuted (paranoia). Hallucinations may be auditory or visual.

Catatonic depression patients have severe psychomotor retardation, engage in excessive purposeless activity, and/or withdraw; some patients grimace and mimic speech (echolalia) or movement (echopraxia).

Peripartum (or postpartum) depression is characterized by onset during pregnancy or in the 4 weeks after delivery. Psychotic features may be present; infanticide is often associated with psychotic episodes involving command hallucinations to kill the infant or delusions that the infant is possessed.

Seasonal pattern depression (aka seasonal affective disorder or SAD) patients experience episodes of major depression at a particular time of year, most often fall or winter. SAD most often happens during the winter months, when the days grow short and it typically goes away in the spring and summer.

Diagnosis begins with the clinical criteria set out above from the DSM-5. Laboratory tests, such as complete blood count, electrolytes, and thyroid-stimulating hormone, vitamin B12, and folate levels are used to rule out physical disorders that can cause depression. Depressive disorders are distinguished from ordinary mood variations based on the presence of significant distress or functional impairment in social, occupational, or other important areas.

Severity is determined by the degree of pain and physical, social, or occupational disability, and by duration of symptoms. Threats or attempt to harm oneself or others are important indicators. Psychosis and catatonia are evidence of severe depression. Melancholic features indicate severe or moderate depression. Coexisting physical conditions, substance use disorders, and anxiety disorders may add to severity.

In addition to treating anxiety associated with depressive disorders, the present invention contemplates the treatment of anxiety disorders with or without depressive symptoms. Anxiety disorders treatable according to the invention include generalized anxiety disorder, panic disorder, phobia-related disorders, separation anxiety disorder, selective mutism, anxiety disorder due to a medical condition, substance-induced anxiety disorder, and other specified anxiety disorder and unspecified anxiety disorder.

People with generalized anxiety disorder (GAD) display at least 3 symptoms of excessive anxiety or worry, most days for at least 6 months. The subject of the fear and anxiety may be any or a combination of personal health, work, social interactions, and everyday routine life circumstances. This can interfere with social interactions, school, work and other aspects of life. GAD symptoms include: feeling nervous, irritable, restless, wound-up, or on-edge, being easily fatigued; having difficulty concentrating; mind going blank; having muscle tension; difficulty controlling feelings of worry; having sleep problems, such as difficulty falling or staying asleep, restlessness, or unsatisfying sleep; feeling weak or tired; having a sense of impending danger, panic or doom; having an increased heart rate; hyperventilation, sweating, and/or trembling; and experiencing gastrointestinal problems.

People with panic disorder have recurrent unexpected panic attacks. Panic attacks are sudden periods of intense fear that come on quickly and reach their peak within minutes. Attacks can occur unexpectedly or can be brought on by a trigger, such as a feared object or situation.

During a panic attack, people may experience: heart palpitations, a pounding heartbeat, or an accelerated heartrate; sweating; trembling or shaking; sensations of shortness of breath, smothering, or choking; feelings of impending doom; and/or feelings of being out of control.

Patients with panic disorder often worry about when the next attack will happen and actively try to prevent future attacks by avoiding places, situations, or behaviors they associate with panic attacks. Worry about panic attacks, and the effort spent trying to avoid attacks, cause significant problems in various areas of the person's life, including the development of phobia-related disorders.

Patients with phobia-related disorders present with an intense fear of—or aversion to—specific objects or situations. The fear patients with phobias feel is out of proportion to the actual danger caused by the situation or object.

Patients with a phobia may have an irrational or excessive worry about encountering the feared object or situation, take active steps to avoid the feared object or situation, experience immediate intense anxiety upon encountering the feared object or situation, endure unavoidable objects and situations with intense anxiety

Phobia-related disorders treatable according to the invention include specific phobias (flying, heights, certain animals, blood, injections), social anxiety disorder, agoraphobia, separation anxiety disorder, selective mutism, anxiety disorder due to a medical condition, substance-induced anxiety disorder, and other specified and unspecified anxiety disorder.

Patients with specific phobias (simple phobias) have an intense fear of, or feel intense anxiety about, specific types of objects or situations. Specific phobias include: flying, heights, specific animals (spiders, dogs, snakes), receiving injections, and blood.

Patients with social anxiety disorder (social phobia) have a general intense fear of, or anxiety toward, social or performance situations. They fear that actions or behaviors will be negatively evaluated by others, leading to embarrassment. This often causes patients to avoid social situations. Social anxiety disorder can manifest in a range of situations or environments (e.g., workplace or school).

Patients with agoraphobia have an intense fear of two or more of the following situations: using public transportation; being in open spaces, being in enclosed spaces; standing in line or being in a crowd; and being outside of the home alone. The patient may think it would be difficult to leave if they have a panic attack, which would be embarrassing, leading the patient to avoid the situation. At its most severe, agoraphobia leaves the patient housebound.

Separation anxiety disorder does not occur just in children; it also occurs in adults. Patients with separation anxiety disorder fear being parted from people to whom they are attached. They often worry that harm come to their attachment FIGURES while separated, leading to avoiding being separated and avoiding being alone. Separation anxiety may result in nightmares about being separated from attachment FIGURES or physical symptoms when separation occurs or is anticipated.

Selective mutism occurs when patients fail to speak in specific social situations despite having normal language skills. It usually occurs in children, before the age of 5, and is often associated with extreme shyness, fear of social embarrassment, compulsive traits, withdrawal, clinging behavior, and temper tantrums. Selective mutism is often diagnosed with other anxiety disorders.

Anxiety disorder due to a medical condition includes symptoms of intense anxiety or panic that are directly caused by a physical health problem. Substance-induced anxiety disorder is characterized by symptoms of intense anxiety or panic that are a direct result of misusing drugs, taking medications, being exposed to a toxic substance or withdrawal from drugs.

Other specified anxiety disorder and unspecified anxiety disorder are terms for anxiety or phobias that do not meet the exact criteria for any other anxiety disorders but are significant enough to be distressing and disruptive to social, occupational or other functioning.

### Dosing Regimens

In accordance with the treatment methods of the present invention, a patient in need of treatment is administered a therapeutically effective amount of a ROCK inhibitor or a pharmaceutically acceptable salt thereof one or more times a day. Generally, the methods are best accomplished with oral administration. The lowest therapeutically effective amount of fasudil, for example, is 1 mg per day, generally administered in 2 to 3 equal portions to obtain the full daily dose. The highest therapeutically effective dose is less than 60 mg per day. One preferred dosing regimen involves the treatment with 5, 10, 15 or 20 mg of fasudil hydrochloride hemihydrate three times per day using an immediate-release formulation, for a total daily dose of 15-60 mg. Preferred dosing exceeds a daily dose of 1 mg per day, with most preferred ranges for daily dosing being 15 mg to 60 mg administered in three equal amounts during the day. Other preferred daily doses will range from 10 mg to 30 mg per day. 15 mg to 50 mg per day, or 35 mg to 50 mg per day. A further dosing regimen involves the treatment with 15 to 30 mg of fasudil hydrochloride hemihydrate only two times per day using an immediate-release formulation, for a total daily dose of 30-60 mg. Based on ROCK inhibitory activity, one skilled in the art can readily extrapolate the provided dosing ranges for fasudil to other ROCK inhibitors.

Certain patient sub-populations, such as renally impaired patients and/or older patients (e.g., 65 or older) may need lower doses or extended release formulations instead of immediate release formulations. Fasudil hydrochloride hemihydrate may have higher steady-state concentrations when given at usual doses to patients with renal disease and lower doses to lower the Cmax or delay the time to Cmax (increase the Tmax) may be required.

Renal dysfunction occurs with age and as the result of numerous disorders, including liver cirrhosis, chronic kidney disease, acute kidney injury (for example, due to administering a contrast agent), diabetes (Type 1 or Type 2), autoimmune diseases (such as lupus and IgA nephropathy), genetic diseases (such as polycystic kidney disease), nephrotic syndrome, urinary tract problems (from conditions such as enlarged prostate, kidney stones and some cancers), heart attack, illegal drug use and drug abuse, ischemic kidney conditions, urinary tract problems, high blood pressure, glomerulonephritis, interstitial nephritis, vesicoureteral, pyelonephritis, sepsis. Kidney dysfunction may occur in other diseases and syndromes, including non-kidney-related diseases that may occur along with kidney dysfunction, for example pulmonary artery hypertension, heart failure, and cardiomyopathies, among others.

Kidney function is most often assessed using serum (and/or urine) creatinine. Creatinine is a breakdown product of creatine phosphate in muscle cells and it is produced at a constant rate. It is excreted by the kidneys unchanged, principally through glomerular filtration. Accordingly, elevated serum creatinine is a marker for kidney dysfunction and it is used to estimate glomerular filtration rate.

Normal levels of creatinine in the blood are approximately 0.6 to 1.2 mg/dl in adult males and 0.5 to 1.1 mg/dl in adult females. When creatinine levels exceed these FIGURES, the subject has renal dysfunction, and is, therefore, treatable according to the invention. Mild renal impairment/dysfunction occurs in the range of 1.2 mg/dl to 1.5 mg/dL. Moderate renal impairment/dysfunction is considered to occur at creatinine levels exceeding 1.5 mg/dL. Severe renal impairment, which includes what is considered to be renal failure, is defined as a serum creatinine level of ≥2.0 mg/dl or the use of renal replacement therapy (such as dialysis). Treating subjects with mild, moderate and severe renal impairment is specifically contemplated.

As indicated, creatinine levels are considered to be a surrogate for glomerular filtration rate (GFR) and serum creatinine levels alone may be used to estimate glomerular filtration rate using the Cockroft-Gault equation.

According to the National Kidney Foundation, the following GFRs indicate the varying levels of renal function:

| GFR | |
|---|---|
| (ml/min/1.73 m²) | Renal Function |
| ≥90 | Normal or high |
| 60-89 | Mildly decreased |
| 45-59 | Mildly to moderately decreased |
| 30-44 | Moderately to severely decreased |
| 15-29 | Severely decreased |
| <15 | Kidney failure |

In general, creatinine clearance (estimated glomerular filtration rate) may be derived directly from serum creatinine using the Cockroft—Gault equation:

$$\\ creatinine clearance = ( ( ( 140 - age in years ) \times ( wt in kg ) ) \times 1.23 ) / ( serum creatinine in mol / L )$$

For women the result of the calculation is multiplied by 0.85.

Empirically measured creatinine clearance may also be used directly as an estimate of glomerular filtration rate by looking at serum creatinine and urine creatinine levels. Specifically, urine is collected over 24 hours and the following equation is applied to ascertain creatinine clearance:

$$\\ Creatinine Clearance ( mL / min ) = Urine Creatinine Concentration ( mg / mL ) * 24 hour urine volume ( mL ) / Plasma Creatinine Concentration ( mg / mL ) * 24 hour * 60 minutes$$

In one embodiment, dose of fasudil for mild to moderate renal impairment is reduced to 50-80 mg per day. In another embodiment, the dose of fasudil is not reduced but is administered one time per day in an extended release dosage form.

In another embodiment, the dose is not reduced for mild to moderate renal impairment.

In one embodiment, the dose of fasudil is reduced to 30-45 mg per day for severe renal impairment. In another embodiment, the dose of fasudil is not reduced but is instead administered one time per day in an extended release dosage form.

In a further embodiment, the dose is reduced where serum creatinine (SCr)>2 and/or an increase in SCr>1.5× from baseline, and/or a decrease in eGFR>25% from baseline.

Patient size is an important factor to consider when using creatinine-based estimates of renal function. The units of drug clearance are volume/time (mL/min), whereas the units of estimated GFR for chronic renal disease are volume/time/standard size (mL/min/1.73 m2). Generally, doses may be adjusted down (e.g., 40-50 mg per day) for smaller patients and up for larger (e.g., 120 mg per day) for obese patients. A smaller male would be about 160 pounds or less. A smaller female patient would weigh about 130 pounds or less. Patients having a Body Mass Index of 30 and higher is considered obese.

In an embodiment, the patient is a male. In another embodiment, the patient is a female. In an embodiment, the patient is an adult. In other embodiments, the patient is an adolescent or a child.

In addition, older patients may need a lower dose at initiation, with a gradual increase to the recommended dose after days or weeks. In another embodiment, older patients may need lower doses for the duration of treatment. The aged population includes the “young old” who are 65-74, the “old” who are 75-84 and the “frail elderly” who are 85 and older. For example, a starting dose of 30 mg per day for two weeks, followed by 40 mg per day for 4 weeks, then by 50 mg per day. Titration may even be warranted up to about 60 mg per day.

Another embodiment involves the treatment with 5-60 mg of fasudil hydrochloride hemihydrate once per day in an extended-release dosage form. Treatment with an extended-release total daily dose of 60 mg fasudil hydrochloride hemihydrate once per day is preferred. It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

Methods of administering compositions according to the invention would generally be continued for at least one day. Some preferred methods treat for up to 30 days or up to 60 days or even up to 90 days or even more. Treatment for more than 60 days is preferred and treatment for at least 6 months is particularly preferred. The precise duration of treatment will depend on the patient's condition and response to treatment. Most preferred methods contemplate that treatment begins after the onset or appearance of symptoms.

Another embodiment involves the treatment with 30-60 mg of fasudil hydrochloride hemihydrate once per day in an extended release dosage form. Treatment with an extended release total daily dose of 60 mg fasudil hydrochloride hemihydrate is preferred.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

Methods of administering compositions according to the invention would generally be continued for at least one day. Some preferred methods treat for up to 30 days or up to 60 days or even up to 90 days or even more. Treatment for more than 60 days is preferred and treatment for at least 6 months is particularly preferred. The precise duration of treatment will depend on the patient's condition and response to treatment.

### Outcomes

The effectiveness of the inventive therapies may be assessed as is well known to those in the art. Hamilton Rating Scale for Depression (HAM-D) is often used as a primary assessment for depression. The 17-item HAM-D scale is used to assess the severity of depression. It is comprised of individual ratings related to the following symptoms: depressed mood, feelings of guilt, suicide, insomnia, work and activities, retardation, agitation, anxiety, somatic symptoms, genital symptoms, hypochondriasis, loss of weight, and insight. Individual items are scored on either a 3-point (0 to 2) or a 5-point scale (0 to 4), with 0=none/absent and 4=most severe. The total score is the sum of individual items, ranging from 0 to 52; where a higher score indicates more depression.

The HAM-D may be analyzed a number of different ways: Change from baseline to a particular time (e.g., day 3, 15, 28 or 42). A meaningful response should be detectable within 15 days and a very quick response might be seen in 3 days. Drug treated groups will show greater decreases from baseline. Direct comparisons of total scores may also be done between groups at a particular time (e.g., day 15), with drug treated groups showing lower scores. It may also be used assess time to response, defined as time from first administration of study drug to the time when ≥50% reduction in HAM-D score from baseline is achieved. Generally, a response will be seen within several weeks of treatment and response in drug groups will be earlier and sustained as compared to controls. It may also be used in a study to evaluate the percentage of drug versus control participants with a remission, defined as HAM-D total score≤7, at a particular time (e.g., days 1, 3, 8, 12, 15, 21, 28, 35 or 42). Drug treated subjects will show a greater remission rate at all time points. The other scales set out below may be similarly used.

Montgomery Åsberg Depression Rating Scale (MADRS) is also commonly used as a primary assessment for depression. The MADRS is a 10-item diagnostic questionnaire used to measure the severity of depressive episodes in participants with mood disorders. Each item is rated on a 7-point scale from 0 (no symptoms) to 6 (symptoms of maximum severity). The total score ranges from 0 to 60 with a higher score indicating more depression.

The MADRS may be analyzed a number of different ways: Change from baseline to a particular time (e.g., day 3, 15, 28 or 42). A meaningful response should be detectable within 15 days and a very quick response might be seen in 3 days. Drug treated groups will show greater decreases from baseline. Direct comparisons of total scores may also be done between groups at a particular time (e.g., day 15), with drug treated groups showing lower scores. It may also be used assess time to response, defined as time from first administration of study drug to the time when ≥50% reduction in MADRS score from baseline is achieved. Generally, a response will be seen within several weeks of treatment and response in drug groups will be earlier and sustained as compared to controls.

The Clinical Global Impression—Severity (CGI-S) is a 7-point Likert scale to rate the severity of the participant's illness at the time of assessment, relative to the clinician's past experience with participants who have the same diagnosis. A participant is assessed on severity of mental illness at the time of rating as 1=normal, not at all ill; 2=borderline ill; 3=mildly ill; 4=moderately ill; 5=markedly ill; 6=severely ill; and 7=among the most extremely ill participants. A typical analysis will involve comparing the overall score between groups at a particular time (e.g., day 15) or comparing the percentage of participants with a CGI-I response between groups at a particular time (e.g., day 15). A CGI-I response may be defined as having a CGI-I score of “very much improved” or “much improved.”

Hamilton Rating Scale for Anxiety (HAM-A) is often used in depression and anxiety studies. The 14-item HAM-A is comprised of a series of symptoms, and measures both psychic anxiety (mental agitation and psychological distress) and somatic anxiety (physical complaints related to anxiety). The HAM-A total score will be calculated as the sum of the 14 individual item scores. The scoring for HAM-A is calculated by assigning scores of 0 (not present) to 4 (very severe), with a total score range of 0 to 56. A typical analysis will involve comparing the overall score between groups at a particular time (e.g., day 15)

Another important assessment not only for depression, but for any drug that acts on the central nervous system is suicidality, which is most commonly evaluated using the Columbia-Suicide Severity Rating Scale (C-SSRS). As discussed, suicidality is present in about half of patients with depression and specific planning or actions in that regard, suggest a high risk of depression. The C-SSRS is a series of six “yes” or “no” questions relating to suicidality: (1) wish to be dead; (2) non-specific suicidal thoughts; (3) active suicidal ideation without a plan or intent to act; (4) active suicidal ideation without a specific plan, but with some intent to act; (5) active suicidal ideation with both a plan and intent to act; and (6) taken any active steps toward committing suicide. An answer of “yes” on any single question suggests a risk and a “yes” on questions 4, 5 or 6 indicates a high risk. Treatments according to the invention do not increase suicidality risk relative to baseline or relative to a control group.

In assessing anxiety and depression, it is also important to assess from a patient perspective impacts on quality of life, disability, and well-being, as this may be helpful to detect distress or functional impairment in social, occupational, or other important areas, indicative of a disorder. A number of patient-reported outcome measures may be used in this regard, including the Short Form-36 (SF-36), the Patient Health Questionnaire (PHQ-9), the Sheehan Disability Scale (SDS), and The World Health Organisation-Five Well-Being Index (WHO-5). Such instruments may be used as described above, comparing changes over time or absolute scores between groups at a particular time. Treated patients will greater improvements over time than untreated patients and will have higher well-being and better functioning than matched, untreated patients.

The SF-36v2 is a 36-item measure including eight health dimensions which are four physical health status domains and four mental health status domains. Scores on each item are summed and averaged (range=0 “worst”-100 “best”). Higher SF-36 scores indicate a better state of health.

The PHQ-9 is a patient-rated depressive symptom severity scale where scoring is based on responses to specific questions. The score is calculated as the sum of the 9 individual item scores. The PHQ-9 total score ranges from 1 to 27 with a higher score indicating more depression.

The Sheehan Disability Scale (SDS) is a 3-item clinician-rated questionnaire used to evaluate impairments in the domains of work, social life/leisure, and family life/home responsibility. All items are rated on an 11-point continuum (0=no impairment to 10=most severe) with the total SDS score ranging from 0 (no impairment) to 30 (most severe)

WHO-5 evaluates positive psychological well-being. WHO-5 consists of 5 questions and each is rated on a 6-point scale. The total score ranges from 0 to 25 (0=worst possible quality of life; 25=best possible quality of life).

### Combination Therapies

The invention further contemplates combination therapy of the foregoing types of patients with at least one rho kinase inhibitor and at least one known antidepressant or anxiolytic and, in particular, those listed below. Combination therapy may be accomplished by dosing each drug separately or dosing them together in a fixed-dose combination (e.g., a ROCK inhibitor and an antidepressant in a single tablet or capsule, as described above). Dosing of antidepressants and anxiolytics will be the same or lower in the combination as doses conventionally used.

Classes of antidepressant drugs, which may be used to treat depression and anxiety, include selective serotonin reuptake inhibitors (SSRIs), serotonin-norepinephrine reuptake inhibitors (SNRIs), serotonin modulators and stimulators, serotonin antagonists and reuptake inhibitors (SARIs), norepinephrine reuptake inhibitors, norepinephrine-dopamine reuptake inhibitors (NDRIs), tricyclic antidepressants (TCAs), tetracyclic antidepressants (TeCAs), monoamine oxidase inhibitors (MAOIs), NMDA receptor antagonists and GABAA receptor positive allosteric modulators.

SSRIs include citalopram (Celexa), escitalopram (Lexapro), fluoxetine (Prozac), fluvoxamine (Luvox), fluvoxamine CR (Luvox CR), paroxetine (Paxil), paroxetine CR (Paxil CR), sertraline (Zoloft), Vilazodone (Viibryd), Vortioxetine (formerly Brintellix; now Trintellix). SNRIs include desvenlafaxine (Pristiq), duloxetine (Cymbalta), venlafaxine (Effexor), venlafaxine XR (Effexor XR), venlafaxine ER (Effexor ER), milnacipran (Savella), and levomilnacipran (Fetzima). TCAs include amitriptyline (Elavil), desipramine (Norpramin), doxepine (Adapin, Sinequan), imipramine (Tofranil), nortriptyline (Aventyl, Pamelor), amoxapine (Asendin), clomipramine (Anafranil), maprotiline (Ludiomil), trimipramine (Surmontil), and protriptyline (Vivactyl, Vivactil). MAOIs include phenelzine (Nardil), selegiline (Emsam), and tranylcypromine (Parnate). GABAA receptor positive allosteric modulators include zuranolone (SAGE-217). TeCAs include maprotiline (Ludiomil), mirtazapine (Remeron, a TeCA) and Mianserin (Norval). Atypical antidepressants include bupropion (Wellbutrin, an NDRI), bupropion XL (Wellbutrin XL, an NDRI), bupropion SR (Wellbutrin SR, an NDRI), bupropion ER (Wellbutrin ER, an NDRI), nefazodone (Serzone), trazodone (Desyrel, Oleptro, a SARI), vilazodone (Viibryd), atomoxetine (Strattera), and vortioxetine (Brintellix).

In addition to the antidepressants, other common anxiolytics include barbiturates, benzodiazepines, carbamates, antihistamines, opioids and sympatholytics (adrenergic modulators), among others.

Benzodiazepines include alprazolam (Xanax), chlordiazepoxide (Librium), clobazepam (Onfi), clonazepam (Klonopin), clorazepate (Tranxene), diazepam (Valium), estazolam (ProSom), flurazepam (Dalmane), lorazepam (Ativan), midazolam (Versed), oxazepam (Serax), prazepam (Centrax), Quazepam (Doral), Temazepam (Restoril), and triazolam (Halcion). Antihistamines include hydroxyzine (Atarax, Vistaril). Non-Benzodiazepines include eszopiclone (Lunesta), zaleplon (Sonata), zolpidem (Ambien), zopiclone (Imovane). Other anxiolytics include Buspirone (BuSpar). Anticonvulsants include carbamazepine (Tegretol), gabapentin (Neurontin), leveteriacetam (Keppra), lamotrigine (Lamictal), pregabalin (Lyrica), tiagabine (Gabitril), topiramate (Topamax), valproic Acid (Depakote), and vigabatrin (Sabril). Beta-Blockers include propranolol (Inderal) and atenolol (Tenormin). Many other beta blockers are marketed, but only these two are indicated for anxiety. Alpha-Blockers include, prazosin (Minipress), clonidine (Catapres), and guanfacine (Tenex). Antipsychotics include aripiprazole (Abilify), olanzapine (Zyprexa), quetiapine (Seroquel), risperidone (Risperdal), ziprasidone (Geodon).

1. A method of treating a patient suffering from a depression disorder, an anxiety disorder, or both, comprising administering a therapeutically effective amount of a rho kinase inhibitor to said patient.

2. A method according to claim 1, wherein the patient has at least one of the following conditions: major depression, persistent depressive disorder, bipolar disorder, premenstrual dysphoric disorder, other depressive disorder, generalized anxiety disorder, panic disorder, phobia-related disorders, separation anxiety disorder, selective mutism, anxiety disorder due to a medical condition, substance-induced anxiety disorder, other specified anxiety disorder, and unspecified anxiety disorder.

3. A method according to claim 2, wherein the patient has major depression or persistent depressive disorder that is further categorized as anxious distress, mixed feature, melancholic, atypical, psychotic, catatonic, peripartum, postpartum, or seasonal pattern.

4. A method according to claim 1 wherein the rho kinase inhibitor is an isoquinoline derivative.

5. A method according to claim 4 wherein the isoquinoline derivative is fasudil, a salt, or a derivative thereof.

6. A method according to claim 4 wherein said derivative is M3.

7. A method according to claim 1 where said treatment continues for at least 6 months.

8. A method according to claim 5, wherein said isoquinoline derivative is administered in a dose of less than 60 mg per day.

9. A method according to claim 8, wherein said dose is administered in three equal portions throughout the day.

10. A method according to claim 9, wherein the total daily dose is between 15 mg and 50 mg.

11. A method according to claim 9, wherein the total daily dose is between 10 mg and 30 mg per day.

12. A method according to claim 8, wherein the total daily dose is administered in a sustained release formulation.
